(19) United States
(12) Patent Application Publication
(10) Pub. No.: US 2004/0063216 A1
(43) Pub. Date: Apr. 1, 2004

(54) Method for detecting biomarkers

(76) Inventor: Iser Lubocki, Ramat Gan (IL)

Correspondence Address: Iser Lubocki, 5 Hai Taib St, Ramat Gan, 52272, IL

(21) Appl. No.: 10/451,746

(22) PCT Filed: Dec. 24, 2001
(86) PCT No.: PCT/US01/49712

(30) Foreign Application Priority Data
Dec. 24, 2000 (IL) 140501
Dec. 25, 2000 (IL) 140502

(51) Int. Cl. G01N 24/00

(52) U.S. Cl. 436/173

(57) ABSTRACT

According to the present invention, there is provided a noninvasive method for rapid detection of biomarkers, including the steps of subjecting biological material to laser beam irradiation of a single wavelength, enhancing the scattered light returned from passage of said excitation light through the biological material, transmitting this scattered light and measuring it, to obtain data which is characteristic of the biological material being tested, comparing the spectral data to reference spectral data obtained from laser irradiation of the same wavelength applied to a known biological sample contaminated with the biomarker being detected, receiving a diagnosis of the presence or absence of biomarkers of the disease in the biological material, and determining the quantitative value of the biomarkers in the biological material. Also provided is a diagnostic tool for detecting the presence of a biomarker in a sample, the tool including a laser beam irradiation device with excitation light of a certain wavelength, an enhancing kit for enhancing the scattered light returned from passage of the excitation light, through the biological material, and a measurement device to obtain data which is characteristic of the biological material being tested, a comparing device for comparing the data to reference spectral data obtained from laser irradiation of the same wavelength applied to a known biological sample that has the biomarker, a quantitator for quantitating the value of the biomarker in the biological material and a display device for displaying the diagnosis.

### BACKGROUND OF THE INVENTION

1. TECHNICAL FIELD

Generally, the present invention relates to a method of detecting biomarkers of disease. More specifically, the present invention relates to a non-invasive diagnostic test for detecting the presence of disease biomarkers.

2. DESCRIPTION OF RELATED ART

Generally, patients are increasingly performing diagnostic tests in the privacy of their own homes. In the last years, some home diagnostic kits have been marketed in pharmacies, newspaper ads, etc.

These kits have enabled consumers to test for pregnancy, ovulation, blood pressure, blood glucose, cholesterol, urine, pH, alcohol levels, drug levels, cancer, and HIV.

Patients and doctors want tests that are rapid, accurate, painless and not expensive. But for the time being they can have either blood tests, which are painful, not rapid and sometimes expensive or they can have the kits mentioned above which are mostly expensive, mostly inaccurate and demand extensive laboratory work from the patient (to mix solutions, wait, mix again etc.)

The introduction of molecular approaches to medical research has led the discovery of biological and biochemical markers, which are increasingly valuable for predicting and preventing diseases.

Currently, laboratories use conservative techniques to detect these biomarkers, for example: chemical assays, immunoassays and recombinant DNA techniques. These assays are labor intensive, time consuming, expensive, and most of them are invasive. Only few of these assays can predict diseases or can be done on animal products, animal derivatives, or contaminated materials as well.

A biomarker is the result of a sub-clinical or clinical event in the body. It is an indicator of disease susceptibility. Ideally, the increased risk for disease, or the disease associated with the presence of the biomarker should be reversible by appropriate medical intervention. Routinely sample materials for biomarker assays include: blood, urine, feces and biopsy tissues. DNA samples are usually derived from white blood cells, and the presence of chemical metabolites is usually determined from blood or urine.

Biomarkers are compounds, or their metabolites, which can be found within the body of a human being, a mammal or an animal product. They can also contaminate any other material, such as: soil, water air and crops. Biomarkers can predict disease risk. Biomarkers have been categorized into three types: biomarkers of susceptibility, biomarkers of exposure and biomarkers of effect. Standardized criteria for the quantitative and qualitative measurement of these markers have previously been established, and the predictive values of each of them are determined by population studies.

At the present time, the Federal Drug Administration (FDA) acknowledges the use of biomarkers in the clinical laboratory to predict disease risk. But there are many diseases for which laboratory biomarkers are not applied yet, for example: Schizophrenia, ALS, Alzheimer, Parkinson, Prenatal delivery, Toxemia, Gastric-carcinoma, Metastatic Melanoma, Transmissible Spongiform Encephalopathy (TSE) and more. There are also many diseases for which laboratory biomarkers exist, but the assays are invasive and not rapid, for example: assays for Bovine Spongiform Encephalopathy (BSE), assays for fetal abnormalities, which are taken from amniotic fluid or maternal blood, or tests for different tumors that demand a biopsy.

Referring to one specific disease, Mad Cow Disease, also known as Bovine Spongiform Encaphalopathy or the Prion disease. This disease doesn't yet have a non invasive rapid test. It swept through Great Britain during the past decade, necessitating the slaughter of entire herds of cattle, resulting in vast economic loss and widespread panic. The pathological agent was shown to be a protein unit termed a prion, for “proteinaceous infectious particle”, which appears as protein plaques in the brains of infected mammals (Prusiner et al., Cell 38, 127-134, 1984). It was speculated that the disease could pass from a bovine host carrying bovine spongiform encephalopathy (BSE) or theoretically from another mammalian host carrying transmissible spongiform encephalopathy (TSE), through the food chain to humans or other animals. It would therefore be useful to develop a test for use in testing animals, as well as animal derivatives and products used commercially in the food and health industries for the presence of those infectious prions, or other biomarkers of this disease.

Prions are the first known instance of a protein unit acting as an infectious agent, which puzzled scientists and increased the level of fear of the disease. Prion disease is manifested in humans by increasing signs of ataxia or dementia and eventual fatality. Brain tissues of humans suffering from one of the three human equivalents of the disease, Creutzfeldt-Jakob Disease (CJD), kuru or fatal familial insomnia (FFI) show histological findings similar to those of their bovine counterparts.

In the prior art, detection of the infectious agent in animals required, for the most part, slaughter of the suspected animal, followed by analysis of the brain tissues, either by histopathology or by chemical or biological assays. U.S. Pat. No. 5,792,901 describes a transgenic laboratory animal that can be inoculated with suspected tissue in order to determine if prion disease develops. In the '901 patent, there is disclosed the use of genetic engineering to make the laboratory animal more susceptible to infection of prions from other species. Detection of prion disease is still lengthy using these animals, taking up to six months.

PrPC, the non-infectious form of prions, is no different in primary structure than PrPSc, the infectious form. This makes early biochemical diagnosis of prion disease, before appearance of symptoms, all the more difficult. The difference between PrPC and PrPSc is thought to lie in their secondary structure, which makes PrPC vulnerable to Proteinase K digestion, while PrPSc is resistant to proteinase digestion. This distinction has been utilized in detection of infectious prions. International Publication No. WO 00/298,498 discloses an immunoassay to detect infectious prions, in which a biological tissue or fluid is treated with Proteinase K or with another reagent to facilitate prion extraction, and a monoclonal antibody sandwich is formed on a solid support. This method requires several hours of laboratory work, and cannot be performed on site when screening is done on cattle.

The need exists for a rapid assay to diagnose prion diseases in mammals without necessitating autopsy of the suspected animal. It is the object of the present invention to provide a method of detecting TSE in mammals that preferably can be performed with a hand-held device on-site, before appearance of symptoms. The present method is relatively non-invasive, since it utilizes bodily fluids or materials, some of which are excreted and easily obtainable. The method is not expensive, not labor-intensive, since the biological material being tested almost need not undergo purification or treatment. The method can be applied as well for detection of infectious prions, or other biomarker of TSE, in animal products and animal derivatives, such as gelatin, which is present in 80% of all commercial drugs. This is an important application at localities in proximity to a source of outbreak, since the disease can be transmissible through the food chain, therefore even animal derivatives can be infected. The method can also be applied to detection of the infectious prion or other biomarker of TSE in any material which is contaminated with this biomarker, weather it is solid, liquid, or gas, such as soil, crops, water or air.

Most of FDA approved biomarkers for diseases can be detected in saliva. Saliva contains inorganic compounds of the usual electrolytes of body fluids, the principal ions being sodium, potassium, chloride and bicarbonate but it also contains lipids such as cholesterol, glucose and creatinine. (Thaysen, Thorn and Schwartz, 1954). It contains almost all of the organic compounds of plasma, such as hormones, proteins, immunoglobulins, enzymes, DNA and viruses and bacteria that can be detected in saliva in trace amounts. (Vining and McGinley, 1985).

Saliva is also an adequate source of DNA analysis and typing in certain forensic settings (Walsh et al, 1992). The DNA bending patterns obtained from saliva where indistinguishable from the patterns obtained from blood or hair from the same individual.

Salivary glands have a high blood flow (Haeckel, 1990). They also have an extensively inter-communicating lymph capillary system that runs along both the gland ducts and the blood vessels (Young and Van-Lennep, 1978). In general, there is correlation between the compositions of saliva and plasma (Ritschel and Thompson, 1983). But the large variations in some constituents of saliva are the result of different collection techniques and flowrates.

The measurement of drugs in saliva was suggested as early as the 1970's as an alternative medium (Gorodetzky and Kullberg, 1974). The major disadvantage of saliva is that many drugs are retained in saliva for a shorter period of time than they are retained in urine. But rapid techniques like Raman spectroscopy or the bio-resonance test as used by the present invention, can over-ride this disadvantage.

But for measurements of drugs and hormones that reflect their availability in saliva over a defined time interval, rather than at a particular moment of sampling (active versus passive measurements), An OralDiffusion-Sink (ODS) device is used for the in situ collection of an ultrafiltrate of saliva. It is disposed in the mouth and continuously accumulates the compounds of interest as they diffuse into the device along a concentration gradient (Wade, 1992; Wade and Haegele, 1991).

Raman spectroscopy is a spectroscopic fingerprint method used to detect and characterize compounds. Raman spectroscopy gives specific information regarding the bonds within molecules, their functional groups and their interaction with the surrounding. It reads the naturally imprinted barcodes of compounds.

Raman spectroscopy is used in molecular investigations of biochemical systems, in geology, in gemology, in the pharmaceutical industry and in materials science, for characterization of electronic materials, polymers, nanoparticles, etc. In prior art it was never used for routine screening of diseases.

There are numerous advantages of the Raman technique over the complementary infrared adsorption spectroscopy or over fluorescence: One can measure in aqueous environments, which is extremely important for biological, biochemical and medical systems. The selection rules (i.e. the sensitivity and selectivity) are different than those applicable in absorption spectroscopy and enable detection of otherwise invisible modes of chemical groups within the molecules. One can gain selectivity by controlling the wavelength of the light which is used, its spatial resolution is significantly better than that of infrared spectroscopy (at least by a factor of 10), one can measure at a distance (using optical fibers) and polarization measurements are simple. This is important in proximity to a biochemical hazard but it can also help in routine medical tests in which there is a necessity to get the diagnosis on spot. On those cases there is a possibility to do the test with a hand held device.

Raman's main disadvantage was its low sensitivity compared to absorption spectroscopy, though often large amplifications can be attained (by resonance Raman, surface enhanced Raman, etc). Nevertheless, using modern technology (laser light sources, holographic rejection filters and matrix detectors) the measurement of a Raman spectrum is straightforward and highly sensitive. In conjunction with a microscope one can map samples with respect to the various chemical components. Nowadays the low intensity is overcome by this modern technology. Another potential disadvantage is the interference by fluorescence, which can be overcome by surface enhanced Raman (SERS).

Surface Enhanced Raman (SERS) is a method by which the Raman spectrum is amplified by many orders of magnitude in specially prepared samples containing silver or gold or cooper nanoparticles. This phenomenon occurs most probably because of electromagnetic waves or resonance. The amplification can be by huge factors ranging from ×1000, through more typical ×100,000 to 1,000,000 fold and up to 100,000,000,000,000 (as demonstrated recently). This imparts to Raman spectroscopy an extremely high sensitivity, on the single molecule level. In addition, SERS usually is very helpful in removing any fluorescence background that is often a great nuisance. Thus, a Raman spectrum may be observed where untreated samples exhibited only broad and rather less characteristic fluorescence. Finally, SERS is often selective and can differentiate between various compounds. Thus, a specific target compound can be observed in a mixture with many other molecules in the ambient.

In the prior art, systemic detection of disease biomarkers with the help of a laser beam or Raman spectroscopy where never done systematically without needles. For doctors, blood tests are the bread and butter for systematic detection of diagnostic biomarkers. U.S. Pat. No. 5,243,983 describes the use of Raman spectroscopy to measure the concentration of D-glucose in ocular aqueous humor of a living being. This method is non-invasive but it forces irradiation of the eye with a laser beam and can be applied just for one biomarker, glucose.

The need exists for a rapid assay to diagnose biomarkers in mammals without necessitating blood sampling, biopsy or autopsy. It is the object of the present invention to provide an inexpensive method of detecting medical biomarkers, which preferably can be performed with a hand-held device on-site, before appearance of symptoms. The method of the present invention is non-invasive, since it utilizes body fluids or materials, some of which are excreted and easily obtainable (for example: saliva). The method is not labor-intensive, since the biological material being tested, mostly need not undergo purification or treatment. The method can be applied as well for detection of infectious biomarkers in animal products and animal derivatives, such as gelatin; which is present in 80% of all commercial drugs. This is an important application at localities in proximity to a source of outbreak, since the disease can be transmissible through the food chain, therefore even animal derivatives in drugs can be infective. The method can be applied as well for detection of biomarkers in other materials which are contaminated by thos biomarkers, such as: soil, crops, water and air. This is an important application at localities in proximity to a bio-chemical hazard. Since some diseases can be transmissible through any material, therefore even soil, crops, water, air or any other material contaminated by the biomarker can be toxic or infectious.

It would therefore be useful to develop a method which can be used for the detection of biomarkers in any material which is a carrier of the biomarkers contaminated by that biomarker

### SUMMARY OF THE INVENTION

In the present invention, the term “biomarker” relates to: organic compounds, hormones, proteins, globulin, hemoglobin, fibrinogen, bilirubin, enzymes, RNA, DNA, blood types, viruses, bacteria, fungi, rickettsia, chiamydia, Prions, parasites, toxins, complements, antigens, antibodies, tumor markers, inorganic compounds, electrolytes, minerals, lipids, fatty-acids, glucose, creatinine, uric acid, urea, cort elements, vitamins, antioxidants, bioflavonoids, herbs, and herbal complexes, fibers, soy, lecithin, probiotics, drugs, drug metabolites, alcohol, biochemical hazards and other foreign chemicals, amino acid sequences, a genetic sequence, a spatial configuration of a protein, carbohydrate, lipid or mineral and specific patterns or specific amounts of ions, molecules or compounds.

In the present invention, the term “biological material” relates to: a biological excretion, and is selected from: saliva, urine, feces, mucous secretions, sweat, tears, milk, semen, blood, vaginal discharge, vaginal bleeding, bodily fluids and exhaled gases, biological tissue or smear selected from tonsils, nasal passages, buccal tissue, third lid tissue, squamous skin cells, nails, hair and hair roots, a part of the mammal and is selected from ear lobe, eardrum, tongue, oropharynx, conjunctiva of the eye, iris, aqueous humor, eye lid, rectum, nostrils, skin, hair or nails, an animal product or derivative, and is selected from serum proteins, hormones, bone meal, animal feed, gelatin, tallow, nutritional supplements, food products, processed food products or animal derivatives, any material which can carry and transmit the biomarker, whether it is solid, liquid, or gas, such as soil, crops, envelopes, water, and air.

In the present invention, the term “disease” relates to: A human being disorder, a veterinary disorder, a mammal disorder, a plant disorder or a simple organism disorder, a nutritional disorder, an infectious disease, an endocrine and metabolic disorder, a gastrointestinal disorder, a hepatic and biliary disorder, a musculoskeletal disorder, a pulmonary disorder, an ear nose and throat disorder, an ophthalmologic disorder, a dental and oral disorder, a dermatological disorder, a hermatologic and oncologic disorder, an immunology disorder, an allergic disorder, a neurological disorder, a psychiatric disorder, a cardiovascular disorder, a genitourinary disorder, a gynecological and obstetric disorder, a pediatrics disorder, a pre natal and embryonic disorder, a disorder due to physical agents, a genetic disorder, a geriatric disorder, an environmental disorder, a biochemical hazards disorder, an occupational disorder, poisoning, or disorder related to drug therapy.

According to the present invention, there is provided a non-invasive method for rapid detection of biomarkers, including the steps of: collecting biological material in a relatively non invasive matter, preparing biological material in a relatively non destructive manner, transporting biological material to a testing facility, subjecting biological material to laser beam irradiation of a single wavelength, enhancing the scattered light returned from passage of said excitation light through the biological material, transmitting this scattered light and measuring it, to obtain data which is characteristic of the biological material being tested, comparing the spectral data to reference spectral data obtained from laser irradiation of the same wavelength applied to a known biological sample contaminated with the biomarker being detected, receiving a diagnosis of the presence or absence of biomarkers of the disease in the biological material, and determining the quantitative value of the biomarkers in the biological material, compiling and displaying the received diagnosis to an end user in a simple illustrative manner. Also provided is a diagnostic tool for detecting the presence of a biomarker in a sample, the tool including a collecting and transporting device, a laser beam irradiation device with excitation light of a certain wavelength, an enhancing kit for enhancing the scattered light returned from passage of the excitation light, through the biological material, a transmitter to transmit the scattered light, and a measurement device to obtain data which is characteristic of the biological material being tested, a comparing device for comparing the data to reference spectral data obtained from laser irradiation of the same wavelength applied to a known biological sample that has the biomarker, a quantitator for quantitating the value of the biomarker in the biological material and a display device for displaying the diagnosis. In accordance with a preferred embodiment of the present invention, there is also provided a method and a tool for rapid and relatively non invasive detection of biomarkers of the Transmissible Spongiform Encephalopathy in mammals, human beings or animal products and derivatives, comprising the steps of collecting biological material in a relatively non invasive matter, preparing biological material in a relatively non destructive manner, transporting biological material to a testing facility, subjecting biological material to laser beam irradiation of a single wavelength, enhancing the scattered light returned from passage of said excitation light through the biological material, transmitting this scattered light and measuring it, to obtain data which is characteristic of the biological material being tested, comparing the spectral data to reference spectral data obtained from laser irradiation of the same wavelength applied to a known biological sample contaminated with the biomarker being detected, receiving a diagnosis of the presence or absence of biomarkers of the disease in the biological material, and determining the quantitative value of the biomarkers in the biological material, compiling and displaying the received diagnosis to an end user in a simple illustrative manner. Also provided is a diagnostic tool for detecting the presence of a biomarker in a sample, the tool including a collecting and transporting device, a laser beam irradiation device with excitation light of a certain wavelength, an enhancing kit for enhancing the scattered light returned from passage of the excitation light, through the biological material, a transmitter to transmit the scattered light, and a measurement device to obtain data which is characteristic of the biological material being tested, a comparing device for comparing the data to reference spectral data obtained from laser irradiation of the same wavelength applied to a known biological sample that has the biomarker, a quantitator for quantitating the value of the biomarker in the biological material and a display device for displaying the diagnosis.

### BRIEF DESCRIPTION OF THE DRAWINGS

Other advantages of the present invention are readily appreciated as the same becomes better understood by reference to the following detailed description when considered in connection with the accompanying drawings wherein:

FIG. 1—SERS of riboflavin in water with silver;

FIG. 2—SERS of E. coli with colloid; and

FIG. 3—Comparison between SERS of riboflavin and SERS of E coli.

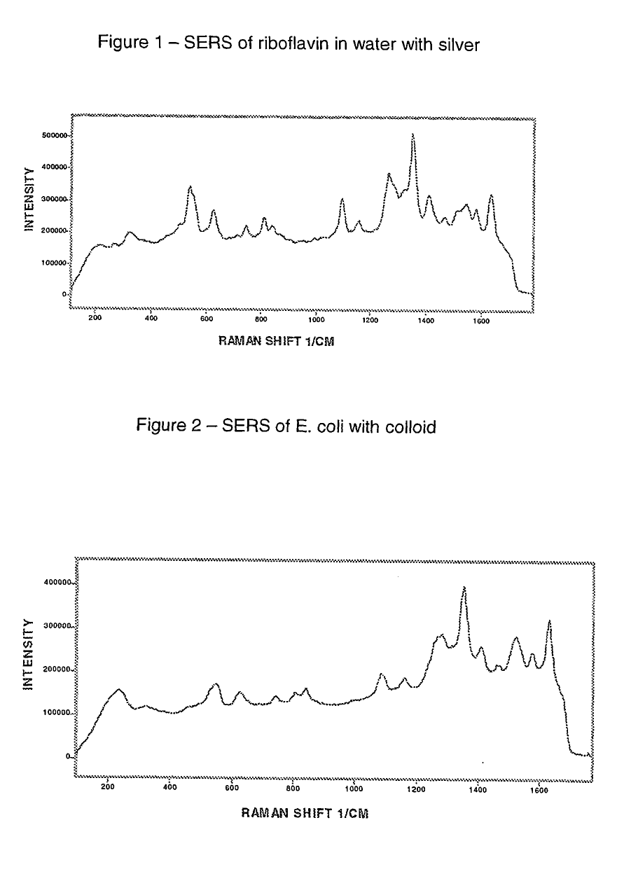

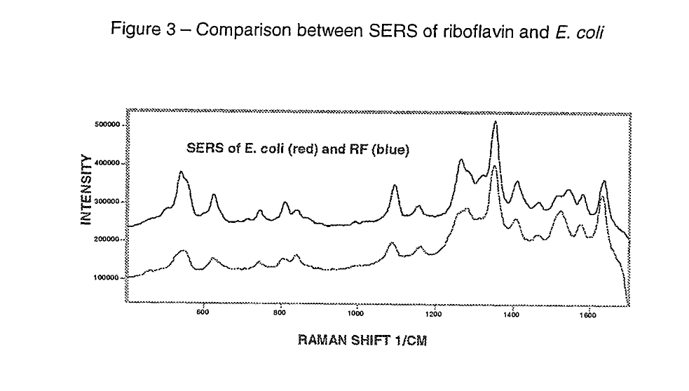

### DETAILED DESCRIPTION OF THE INVENTION

Generally, the present invention provides a method and a diagnostic tool for use in detecting biomarkers which exist in a biological material. The key attributes of the method and the diagnostic tool allows for the non-invasive and non-destructive analysis of solids, liquids, and gases for the presence and quantity of biomarkers of diseases. It also provides for one-micrometer spatial resolution when coupled to a microscope. Further, the method and tool enable rapid sample identification, in typically less than 60 seconds. The method and tool are easy to use, with minimal sample preparation required and can include automatic mapping of samples and automatic displaying of results. In one embodiment of the present invention there is provided remote testing possibilities with fiber optic probes and a possibility for connection to a remote diagnostics center through the Internet. In another embodiment of the present invention there is provided a bioresonance device with an enhancing kit that increases the sensitivity of the test (SERS and SERRS).

The term “biological material” relates to a biological tissue or a biological fluid sample, feces, or a product made from animal tissues, organs or secretions. For example, the biological material can be selected from saliva, urine, feces, mucous, secretions, sweat, tears, milk, semen, vaginal discharge and vaginal bleeding, bodily fluids, exhaled gases. The biological material can be a biological tissue or smear selected from tonsils, nasal passages, buccal tissue, third lid tissue, squamous skin cells, nails, hair and hair roots. The biological material can also part of the mammal and is selected from ear lobe, eardrum, tongue, conjunctiva of the eye, or rectum. The biological material can also be an animal product or animal derivative such as: gelatin and tallow. In the present invention, the term “biological material” relates also to any material which can be contaminated or can carry and transmit an infective or toxic biomarker, whether it is solid, liquid, or gas, such as ground, crops, water, and air.

In one embodiment of the present invention, a biological sample is obtained non invasively from a mammal, possibly from a cow or a human. The biological sample need not be a brain tissue, rather it can be a body fluid or tissue, and so the mammal need not be slaughtered or diagnosed posthumously as the biological sample can be taken non-invasively.

The biomarkers detected in the present invention in mammals or in animal products are biomarkers known to the show the exposure, effect, existence or susceptibility of a disease such as: organic compounds, hormones, proteins, globulin, hemoglobin, fibrinogen, bilirubin, enzymes, RNA, DNA, blood types, viruses, bacteria, fungi Rickettsia, Chlamydia, Prions, parasites, toxins, complements, antigens, antibodies, inorganic compounds, electrolytes, minerals, lipids, essential fatty-acids, glucose, creatinine, uric acid, urea, cort elements, vitamins, anti-oxidants, bioflavonoids, herbs and herbal complexes, fibers, soy, lecithin, probiotics, modulators, drugs, drug metabolites, alcohol, biochemical hazards and other foreign chemicals. The biomarker can also show the presence of an amino acid sequence, a genetic sequence, a spatial configuration of a protein, carbohydrate, lipid, mineral or a special configuration or amount of ions, molecules, compounds, or infectious agents.

The markers can also be specific patterns of sulfur bridges or a molecular structure containing copper ions, manganese ions, antioxidants (such as Sod and low molecular weight anti oxidants—LMWA), radical scavengers (such as caratinoids and vitamin E), heat shock proteins (HSP70, HSP104, GROEL), reactive oxygen species (ROS), spiroplasma derivatives, acino-bacteria, bacteria, fungi Rickettsia, Chlamydia, Prions, parasites, viruses, apolipoprotein E, Corticoid, or Nitric oxide synthetase. The markers are correlated with the presence of the infectious agent, and can be selected from, but are not limited to: an amino acid sequence, a genetic sequence, a spatial configuration of beta sheets and α-helices within a protein or a special configuration or amount of ions, molecules, or compounds.

The method also involves the interaction of light with matter. Incident laser light causes bends between atoms to vibrate. Analysis of scattered light, as a Raman spectrum (which is the plot of intensity of scattered light vs. energy difference), reveals information about samples, chemical structures and physical state.

In the present invention, the biological material, with the investigated biomarker, is exposed to a laser source. The laser source can be a mobile one, having limited capacity and emission of short pulses of 10-15 seconds, such as a laser diode which transmits in a limited spectral range, or an adjustable laser titanium sapphire diode, a continuous wave laser diode, a neodymium doped yttrium aluminum gamet laser (Nd-YAG laser), an optical parametric oscillator, gas ion lasers, solid state lasers, allowing emission of light of various wavelengths.

In a preferred embodiment of the present invention, In order to determine the presence of a biomarker the Raman spectroscopic fingerprint that is generated from mammal, or from the animal product, or from the contaminated material is enhanced by using a bio-resonance device.

Bio information energy research has found that all matter has its own unique vibratory signal. Photon and electric beams can also carry this information. It is now known that every human being, animal and material has its own fingerprint of bio-resonance. To record this bio-resonance data, a bio-resonance device was used.

A bio-resonance device is a device which applies light, resonant and non-resonant, with the analyzed molecules and/or that enhances the light scattering by metal surfaced enhancement in resonant and off resonant processes. This technique can produce extremely sensitive analysis of ultra low concentrations by employment of either of the two or both additions to normal Raman, namely resonance and surface enhancement.

According to theories in the field of bioresonance, all matter has its own unique resonant “fingerprint” signal, which can be carried by electromagnetic waves (light). Surface enhancement occurs when a molecule is absorbed onto a metal surface such as silver, gold or copper and the excited. Resonance is achieved by using a colored molecule that has a chromophore coincident with the excitation wavelength of light used for inducing the Raman scattering. Absorption of chromophore onto a metal surface results in surface enhanced resonance Raman scattering (SERRS).

Pigmented proteins can be used as chromophores. Examples of such chromophores include, but are not limited to, metalloporphyrins, carotenoides, fullerenes, polydiacetylenes, fluorophores such as Hex and Rhodamine, and also the prion and other exotic molecules and biomarkers that strongly absorb in the visible.

In a preferred embodiment of the present invention, in addition to the Raman spectroscopic fingerprint, a surface enhanced resonant Raman fingerprint (SERRS) or a surface enhanced Raman fingerprint (SERS) is generated from the mammal or from the animal product, to determine presence of infectious prions, or other biomarkers.

One application of the method disclosed in the present invention is as a blood or saliva test for diagnosis of TSE in mammals or humans. Presently, a cow or a human being presenting symptoms of ataxia or dementia suspected of having TSE can only be positively diagnosed post-mortem or by performing a brain biopsy. Prompt diagnosis can lead to prompt prevention of transmission to others and to prompt treatment. In humans and also in other mammals or transmissible materials, the test can be performed in a laboratory, in the vicinity of one, or even at the point of care, and so the laser light source and other equipment can be mobile.

In a preferred embodiment a saliva sample is obtained in the field from a cow. It is exposed to a bioresonance device in order to enhance the spectral data achieved. The saliva sample is subjected to Raman spectroscopy using a handheld laser light source; set to emit light at a predetermined wavelength shown to cause vibration in biological samples taken from carriers of BSE. The light is scattered upon passage through the enhanced saliva sample, and the spectral data is recorded using a spectrometer or a similar system for spectral analysis. The spectral data obtained is transmitted via the Internet to a remote laboratory. In the laboratory it is analyzed and compared with “known” reference spectral data from a library of spectral data of prions or BSE markers. The reference spectral data was previously obtained by performing Raman spectroscopy, or super enhanced Raman spectroscopy, or super enhanced resonance Raman spectroscopy on saliva from a cow that was a known carrier of the infectious agent—the abnormal prion, PrPSc or any other TSE marker. If the spectral “fingerprint” of both is identical, the cow being tested is diagnosed as a carrier of BSE. The diagnosis is received minutes after the saliva sample was taken, a great advantage over what is accepted in prior art—hours or months later. The diagnosis is received while the cow is still pre-symptomatic.

Most of all, the diagnosis is received non invasively without slaughtering the cow or without performing brain biopsy, tonsillectomy or appendectomy as was done in prior art.

The use of saliva samplings as non-invasive qualitative and quantitative techniques is promising. Being readily accessible and collectable, saliva can show many advantages over “classical” biological fluids such as blood and urine. New techniques for analyzes of saliva, as for identifying the components affecting drug concentrations in saliva, are increasingly important.

Another application of the method disclosed is a saliva test for vitamins in mammals. Vitamins are very important for our health. This is established by the accessibility of vitamin in the pharmacy and grocery stores.

A deficiency or excess of a vitamin can cause various diseases susceptibility or can potentially lead to death. So it is important to test their existence in mammals non-invasively. This can be done by a simple saliva test for vitamins. As an example, a Raman spectroscopy for vitamin B is described below.

The target molecule is vitamin B₂ (riboflavin) and its related compound flavin adenine dinucleotide (FAD). This vitamin and FAD are extremely important compounds in living organisms, and participate in many life-sustaining processes.

The aqueous medium here simulates saliva (which main constituent is water), and bacteria was used to simulate a most complex and demanding biochemical and biological environment.

The samples were treated for SERS by silver to demonstrate the capabilities of this unique method of detecting and characterizing compounds by Raman spectroscopy.

The spectrum of vitamin B₂ in water without any treatment with silver shows a very broad and non-specific (fluorescence) signal and one cannot use this signature to identify the vitamin.

The first figure shows the SERS spectrum of this vitamin in water treated with silver. An intense and highly specific fingerprint (“molecular barcode”) is observed that can be attributed unambiguously to riboflavin. No other molecule has the same spectrum. The broad fluorescence background has been almost totally removed.

One sees a spectral landscape consisting of “hills” of various heights. The spectrum is characterized by the position of these “hills” (the Raman shift) and their relative heights. One can apply additional discrimination tools (such as polarization components) but they are usually unnecessary for compound identification. The second figure shows the silver SERS spectrum of Escherichia Coli bacteria (a common intestine bacteria).

A very clear and intense Raman spectrum is observed (while untreated bacteria show only a broad, featureless spectrum). This demonstrates the amplification of the SERS and the very effective reduction of the fluorescence. The spectrum observed is practically identical to that of vitamin B₂ in water. Thus, of all the many compounds and biochemicals present in the bacteria cell, the signal of a minority compound, the vitamin B₂, was picked up. The specificity of this method can be tailored to different compounds by varying the silver-treatment protocols, by changing the laser used in the measurement, as well as by performing prescribed chemical pretreatments.

In the next figure the SERS spectrum of riboflavin, B₂, and that of the silver treated E. coli bacteria were placed together to demonstrate their great similarity and the unambiguous identification of the compound observed in the bacteria.

In summary, there was demonstrated that Raman spectroscopy with surface enhancement is capable of detecting and unambiguously identifying a biochemical in a complex environment and in a saliva-like aqueous solution. The SERS variant of this spectroscopy provides extremely large amplifications, highly effective reduction of fluorescence and compound selectivity and specificity.

More specifically, the present invention also provides a non-invasive method and diagnostic tool for rapid detection of biomarkers; these biomarkers can be markers of disease such as transmissible spongiform encephalopathies, in mammals or markers of many different disorders in human beings, animals and plants, such as: A human being disorder, a veterinary disorder, a mammal disorder, a plant disorder or a simple organism disorder, a nutritional disorder, an infectious disease, an endocrine and metabolic disorder, a gastrointestinal disorder, a hepatic and biliary disorder, a musculoskeletal disorder, a pulmonary disorder, an ear nose and throat disorder, an ophthalmologic disorder, a dental and oral disorder, a dermatological disorder, a hermatologic and oncologic disorder, an immunology disorder, an allergic disorder, a neurological disorder, a psychiatric disorder, a cardiovascular disorder, a genitourinary disorder, a gynecological and obstetric disorder, a pediatrics disorder, a pre natal and embryonic disorder, a disorder due to physical agents, a genetic disorder, a geriatric disorder, an environmental disorder, a biochemical hazards disorder, an occupational disorder, poisoning, or disorder related to drug therapy.

There can be three kinds of biomarkers: biomarkers of susceptibility, biomarkers of exposure and bio markers of the disease effect. There are plenty of biomarkers such as: organic compounds, hormones, proteins, globulin, hemoglobin, fibrinogen, bilirubin, enzymes, RNA, DNA, blood types, viruses, bacteria, fungi, rickettsia, chlamydia, Prions, parasites, toxins, complements, antigens, antibodies, tumor markers, inorganic compounds, electrolytes, minerals, lipids, fatty-acids, glucose, creatinine, uric acid, urea, cort elements, vitamins, antioxidants, bioflavonoids, herbs, and herbal complexes, fibers, soy, lecithin, probiotics, drugs, drug metabolites, alcohol, biochemical hazards and other foreign chemicals, amino acid sequences, a genetic sequence, a spatial configuration of a protein, carbohydrate, lipid or mineral and specific patterns or specific amounts of ions, molecules or compounds.

In the present invention the method includes collecting and transporting the biolocial material to the testing facility, in such a manner that will preserve the material and even concentrate it and enhance its spectral data (as described below) and also subjecting biological material from a mammal to irradiation with excitation light of a single wavelength, preferably within the range of 210 to 1500 nanometers, emitted by a laser light source. Next, the method includes enhancing the scattered light returned from passage of the excitation light, through the biological material and measuring the plot of intensity vs. the energy differences and optionally, the polarization, to obtain data which is characteristic of the biological material being tested. The data is then compared to spectral data obtained from a known biological sample infected with transmissible spongiform encephalopathy, or other disease. The diagnosis of the presence or absence of the disease marker in the biological material is determined. Finally, a quantitative value of the biomarker in the biological material is calculated and the result is displayed to the end user in an illustrative and simple manner.

The laser source can be a mobile one, having limited capacity and emission of short pulses of 10 by the power of −15 seconds, such as a laser diode which transmits in a limited spectral range, or an adjustable laser titanium sapphire diode, a continuous wave laser diode, a neodymium doped yttrium aluminum gamet laser (Nd-YAG laser), an optical parametric oscillator, gas ion lasers, solid state lasers, allowing emission of light of various wavelengths. Preferably, the light is of a single wavelength within the range of 210 to 1500 nanometers, emitted by a laser light source.

The enhancing step requires enhancing the scattered light. Enhancement can occur by exposing the biological material to a close proximity of an enhancing substance. An enhancing substrate can include, but is not limited to, a rough surface, metals such as silver, gold or copper, choloids, silica, chromophores, and combinations thereof.

Alternatively, the enhancement can occur using a transfer kit. The transfer kit can be a transparent tube or bag, it includes a nanosize layer of metals such as: gold, silver, copper or aluminum. It can also include choloids, silica, calcium and chromophores such as: metalloporphyrins, carotenoides, fullerenes, polydiacetylenes, fluorophores Hex and Rhodamine and combinations thereof. The tube is made rough so that biological material can be absorbed to get maximal enhancement. The biological material is placed in or on the exterior of a tube, preferably in the field. The tube can then be either scanned and the information can then be transmitted either via fiber optics or the Internet or the tube itself can be transmitted to a remote laboratory, wherein the laboratory sends back the results. In the kit, the biological material is stored in such a manner as to able to be transported. For example the material can be dried or frozen in any manner known to those of skill in the art. For example, the material can be dried with a hair dryer. Instead of drying the material the material can be placed on a filter paper, on a film or on a special rough stone, known to those of skill in the art. The filter paper or film must be able to store the material without adversely affecting the any of the properties of the material. Preferably, the tube, filter paper or film are transparent and can be dried.

In another embodiment of the present invention, the bioresonance device has a nanosize amount of silver, gold, copper or combinations thereof on the edge of the fiber optics. The device functions such that when light comes to the fibers at end of fibers there is enhancement of light that comes into the fiber optics.

Additionally, in accordance with a preferred embodiment of the present invention, measuring the wavelength, intensity and optionally, the polarization of light returned after passage of laser light through said biological sample is performed using one of the following: a spectrometer, a monochrometer or an interferometer.

The present invention provides a method for detection of biomarkers of diseases in mammals, utilizing data obtained from Raman spectroscopy performed on a biological sample to detect the presence or absence of markers of the investigated disease and determine them quantitatively.

In a preferred embodiment of the present invention, the method measures the Raman spectroscopic fingerprint that is generated from a biological material in order to determine the presence of a biomarker using a bio-resonance device. This can determine the risk for the disease for a human being, animal or even a plant.

A bio-resonance device is a device which applies light, resonant and non-resonant, with the analyzed molecules and/or that enhances the light scattering by metal surfaced enhancement in resonant and off resonant processes.

This technique can produce extremely sensitive analysis of ultra low concentrations by employment of either of the two or both additions to normal Raman, namely resonance and surface enhancement.

Raman spectroscopy can be applied for identification of components present in biological material. The principle of Raman spectroscopy is that when single wavelength light, usually emitted from a laser light source, is focused on a biological sample, it causes scattering of a small fraction of energy in the form of light at a different, shifted wavelength (usually lower than the frequency of the incident photons) dependant upon the components of the biological material. This spectral data obtained represents therefore a “fingerprint” which is typical, or characteristic of the specific biological sample, a surface enhanced resonant Raman fingerprint (SERRS) or a surface enhanced Raman fingerprint (SERS) is generated from the mammal or from the animal product, to determine presence of infectious prions, or other biomarkers. The spectral data of a biological material with unknown components can be compared with spectral data of a known biological sample, and thus the unknown components can be identified. The plot of intensity of scattered light versus energy difference is a Raman spectrum of the biological sample, and is dependant upon the components of the biological material. The present invention provides a method for detection of markers of transmissible spongiform encephalopathies, or other disease, in mammals, utilizing data obtained from Raman spectroscopy performed on a biological sample to detect the presence or absence of markers of the disease.

The light scattered upon passage through the saliva sample is enhanced, and the spectral data is recorded using a spectrometer or a similar system for spectral analysis. The spectral data obtained is transmitted via the Internet to a remote laboratory. In the laboratory it is analyzed and compared with “known” reference spectral data from a library of spectral data of the same disease biomarkers.

The reference spectral data was previously obtained by performing Raman spectroscopy with or without super-enhanced spectroscopy or resonance on saliva from a patient that was a known carrier of the same disease or is known to be susceptible to the disease. If the spectral “fingerprint” of both is identical, the patient being tested is diagnosed as having the disease or as susceptible to the disease. The diagnosis is received minutes after the saliva sample was taken a great advantage over what is accepted in prior art—hours or days later. The diagnosis can be received while the patient is still pre-symptomatic. The diagnosis can be delivered immediately to the doctor's clinic or the patient home via the Internet.

According to the present invention there is provided, a mean of comparison is used which is capable of comparing the data obtained from this method. The comparing device is preferably a foreign compound with a pre known Raman spectra and concentration which is used as a comparison baseline.

Also provided is a quantitating device of the present invention includes any device which is able to quantitate the data obtained from the comparing step. Any device known to those of skill in the art as being able to perform this function can be used.

The method and tool of the present invention can also include a compiling device that is capable of compiling the information obtained from the method of the present invention. The information is compiled in such a way as to enable a person, not skilled in the art, to understand the results of the testing. In other words, the information is set forth simply to show a person the results of the method. This information can then be delivered to the end user, who can be a patient, doctor or other person in need of such information via the Internet, wireless devices, or other mechanisms known to those of skill in the art.

The present invention can also include the step of transmitting the biological material or the special data obtained from the biological material for analysis at a remote physical location. For example, saliva can be collected in a transparent kit. This kit includes some rough enhancing material (such as silver, gold or copper). The kit can be transmitted to a remote laboratory, in which it is checked by Raman Spectroscopy. This transmission can be via radio waves, the Internet, fiber optically, wirelessly, or in any other manner known to those of skill in the art.

In the present invention, a biological sample is obtained from a mammal possibly from a human. The biological sample can be taken from the patient without needles or biopsy or surgery. For example, in a preferred embodiment, a saliva sample of a patient is obtained in the doctor's clinic or at the patient's home. The saliva sample is subjected to Raman spectroscopy using a handheld laser light source; set to emit light at a predetermined wavelength in biological samples taken from carriers of the disease investigated.

This method can be used for rapid detection of biomarkers of the infectious agent of a disease, such as transmissible spongiform encephalopathies, in an animal product. The animal product is selected from serum proteins or hormones. The animal product can be an animal derivative selected from bone meal, animal feed, gelatin, tallow, nutritional supplements, food products for human consumption, processed food products for human consumption, animal derivatives used in the food, health, or pharmaceutical industries.

The method can also be used for detecting the biomarkers of the infectious agent of transmissible spongiform encephalopathies, which are abnormal prion proteins, known as PrPSc or Isoform and derivatives of PrPSc, or any other biomarker of any other disease, in mammals or in animal products. The method can also be applied for detection of the biomarkers in any material that is a carrier of the biomarkers whether it is solid, liquid or gas such as: soil, crops, envelopes, water or air. This is an important application in the detection of biochemical hazards since a disease and its biomarker can be transmissible through solids, liquids, or gases.

Throughout this application, various publications, including United States patents, are referenced by author and year and patents by number. The disclosures of these patents in their entireties are hereby incorporated by reference into this application in order to more fully describe the state of the art to which this invention pertains.

The invention has been described in an illustrative manner, and it is to be understood that the terminology, which has been used, is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

1. A method for rapid detection of biomarkers of diseases, comprising the steps of:
subjecting biological material to laser beam irradiation of certain wavelength;
enhancing the scattered light returned from passage of said excitation light, through the biological material;
measuring the scattered light returned, to obtain data which is characteristic of the biological material being tested;
comparing the spectral data to reference spectral data obtained from laser irradiation of the same wavelength applied to a known similar biological material which is a carrier of the biomarker being detected;
receiving a diagnosis of the presence or absence of the biomarkers of the disease in the biological material; and
determining the quantitative value of the biomarkers of the disease in the biological material.

2. The method for rapid detection of biomarkers according to claim 1, wherein said enhancing step can be done before or after subjecting the biological material to a laser beam

3. The method for rapid detection of biomarkers according to claim 1, wherein said enhancing step includes subjecting the biological material or the light scattering to metal surfaced enhancement in a resonant and off-resonant enhancement.

4. The method for rapid detection of biomarkers according to claim 1, wherein the said transmitting step includes transmission of the biological material, or the spectral data obtained from the biological material for analysis at a remote physical location.

5. The method for rapid detection of biomarkers according to claim 1, further comprising measuring the plot of intensity of scattered light versus energy difference, returned after passage of laser light through said biological sample is performed using at least one selected from the group consisting of a spectrometer, a monochrometer, and an interferometer.

6. The method according to claim 1, wherein said method is used for measuring Transmissible Spongiform Encephalopathy in a sample.

7. A diagnostic tool for detecting the presence and quantitative value of a biomarker in a biological material, said tool comprising:
enhancing means for metal surfaced enhancement with resonant and off-resonant enhancement or with chromophore enhancement.
a laser beam irradiation device with excitation light of a certain wavelength;
transmitting means for transmitting the scattered light returned from passage of the excitation light through the biological material;
measuring means for measuring the wavelength to obtain data which is characteristic of the biological material being tested;
comparing means for comparing the data to reference spectral data obtained from laser irradiation of the same wavelength applied to a known similar biological sample that has the same biomarker; in order to obtain a diagnosis of the presence or absence of the biomarkers of the disease in the biological material;
quantitative means for quantifying the value of the biomarker in the biological material.

8. The tool according to claim 7, further including a collecting and transporting device which is preferably a transparent kit with a rough surface or a special stone, in which the biological material is stored in a manner which enables it to be concentrated, preserved and transported.

9. The tool according to claim 7, wherein said laser device is selected from an adjustable laser titanium sapphire diode, a continuous wave laser diode, a neodymium doped yttrium aluminum gamet laser (Nd-YAG laser), an optical parametric oscillator, gas ion lasers, and solid state lasers.

10. The tool according to claim 7, wherein said laser beam has a wavelength within the range of 210 of 1500 nanometers.

11. The tool according to claim 7, wherein said enhancing means is a compound selected from the group consisting essentially of silver, gold, copper, other metals, chromophores such as: metalloporphyrins, carotenoides, fullerenes, polydiacetylenes, fluorophores Hex and Rhodamine and combinations thereof, and is placed in close proximity to said biological material.

12. The tool according to claim 7, further comprising transmitting means for transmitting said data obtained from said biological material for analysis at a remote physical location, consisting of: fiber-optics, Internet, wirelessly or radio waves.

13. The tool according to claim 7, wherein said measuring means is selected from the group consisting essentially of a spectrometer, a monochrometer, and an interferometer.

14. The tool according to claim 7, wherein said comparing means is a comparing device which is capable of comparing the data obtained from the measurement such as a computer.

15. The tool according to claim 7, wherein said quantitating mean is a foreign compound with a pre known Raman spectra and concentration which is used as a comparison baseline.

16. The tool according to claim 7, further comprising compiling and displaying means for compiling, displaying and delivering said data to an end user in a simple illustrative manner.

17. The tool according to claim 7, wherein said tool is a mobile hand-held device.

18. A method for detecting biomarkers, comprising the steps of:
subjecting biological material to laser beam irradiation of a certain wavelength and detecting at least one biomarker for a disease by obtaining data which is characteristic of the biomarker.
